(19) United States
(12) Patent Application Publication
(10) Pub. No.: US 2026/0348952 A1
(43) Pub. Date: Nov. 5, 2026

(54) Nanomaterials Integrated with Microfluidic Paper-Based Analytical Devices for Enzyme-Free Glucose Assay

(71) Applicant: Trustees of Tufts College, Medford, MA (US)

(72) Inventors: Kawin Khachornsakkul, Malden, MA (US); Sameer Sonkusale, Lincoln, MA (US)

(21) Appl. No.: 19/158,937

(22) PCT Filed: Feb. 22, 2024
(86) PCT No.: PCT/US2024/016864

§ 371 Date: Aug. 22, 2025

Related U.S. Application Data
(60) Provisional application No. 63/447,746, filed on Feb. 23, 2023.

(51) Int. Cl. G01N 33/543; G01N 21/78; G01N 33/66

(52) U.S. Cl. CPC G01N 33/54346; G01N 21/78; G01N 33/54386; G01N 33/66

(57) ABSTRACT

A microfluidic paper-based analytical device includes a sampling zone, a detection zone, and a transport channel extending therebetween, all of which comprise paper. A nanozyme and a reagent are disposed on the sampling and detection zones, respectively, with each comprising nanoparticles of first and second metals respectively. The nanoparticles of the second metal are selected to change to a color that corresponds to concentration of an analyte deposited onto the sample zone.

### RELATED APPLICATIONS

This application claims the benefit of the priority date of U.S. Provisional Application 63/447,746, which was filed on Feb. 23, 2023.

### FIELD OF INVENTION

The invention relates to quantitative analysis and in particular to quantitative analysis using nanoparticles.

### BACKGROUND

Cells rely a great deal on glucose as an energy source. Its presence, and in particular in the correct amounts, is vital in primary catabolic cycles for oxidative phosphorylation and glycolysis to create glycogens, proteins, and lipids. The ability to monitor blood glucose levels is thus important. It is therefore of the utmost significance to have a simple, rapid, low cost yet reliable device for monitoring glucose levels.

Known devices for monitoring glucose rely on enzymatic sensors, such as glucose oxidase, glucose dehydrogenase, or horseradish peroxidase.

A difficulty that arises when relying on enzymes is that of stability. The quaternary structure of a folded enzyme is not entirely stable when exposed to variations in temperature, humidity, and acidity. As a result, the shelf life of sensors that rely on such enzymes is limited.

Some efforts have been made to develop non-enzymatic glucose sensors. However, glucose lacks a convenient chromophore, thus making its optical detection difficult. In addition, there is a potential for such sensors to sense other saccharides. Finally, known non-enzymatic glucose sensors do not provide a result in a suitably short time.

### SUMMARY

In one aspect, the invention features an assay device made of paper. The assay device includes a sampling zone, a detection zone, and a transport channel extending therebetween to support capillary flow between the sampling zone and the detection zone, a nanozyme on the sampling zone, and a reagent on the detection zone, The sampling zone, the detection zone, and the transport channel all comprise the paper. The nanozyme and the reagent comprise nanoparticles that comprise first and second metals, respectively. The nanoparticles of the second metal change to a color that corresponds to concentration of an analyte deposited onto the sample zone.

In some embodiments, the reagent is configured to react with a product of a reaction that was catalyzed by the nanozyme, wherein the analyte was a reagent in the reaction.

Other embodiments include a sensor that detects the color and provides an estimate of a concentration of the analyte based on the color. An example of such a sensor is a smartphone having a camera, the smart phone having been configured to carry out colorimetric analysis of an image captured by the camera, for example by executing a suitable application or software.

In some embodiments, the nanozyme comprises metallic nanoparticles that catalyze a reaction with glucose and wherein the reagent comprises metallic nanoparticles that react with a product of the reaction.

In other embodiments, the analyte is glucose, the nanozyme catalyzes oxidation of the glucose, and the reagent reacts with a product of the oxidation.

Still other embodiments include those in which the detection zone is one of a plurality of detection zones and the transport channel is one of a plurality of channels, each of which connects the sampling zone to a corresponding one of the detection zones.

In some embodiments, the transport path is a meandering transport path.

Embodiments further include those in which the nanozyme comprises gold nanoparticles, those in which the reagent comprises silver nanoparticles, those in which the analyte is glucose, and those in which analyte is glucose, the nanozyme comprises gold particles, and the reagent comprises silver nanoparticles.

Also among the embodiments are those in which nanozyme comprises platinum nanoparticles, those in which the reagent comprises nickel nanoparticles, and those in which the reagent comprises nickel-ferrite nanoparticles.

Also among the embodiments are those in which the nanozyme comprises gold nanoparticles that promote oxidation of glucose into gluconic acid and hydrogen peroxide. In such embodiments, the hydrogen peroxide and the reagent interact to cause a color change that depends on glucose concentration.

In another aspect, the invention features a process for using the foregoing products. Such a process, or equivalent, method, includes placing a sample of a bodily fluid on the sampling zone, thereby initiating a reaction that is catalyzed by the nanozyme and causing a reaction product thereof to engage in capillary flow along the transport channel towards the detection zone, wherein the reaction product interacts with the reagent at the detection zone to cause a color change. The method further includes estimating a concentration of the analyte in the sample, for example, by carrying out colorimetric analysis to determine a change in color at the detection zone.

In another aspect, the invention features a process that is specially adapted for the manufacture of the foregoing product. Such a process, or equivalent, method, includes the use of a wax printer to print, on a piece of the paper, the sample region, the detection region, and the transport channel. The process continues with pre-loading the sample region with the nanozyme and pre-loading the detection region with the reagent.

These and other features of the invention will be apparent from the following detailed description and the accompanying figures, in which:

### BRIEF DESCRIPTION OF THE FIGURES

FIG. 1 shows a microfluidic paper-based analytical device and

FIG. 2 shows steps in the manufacture and use of the device in FIG. 1.

### DETAILED DESCRIPTION

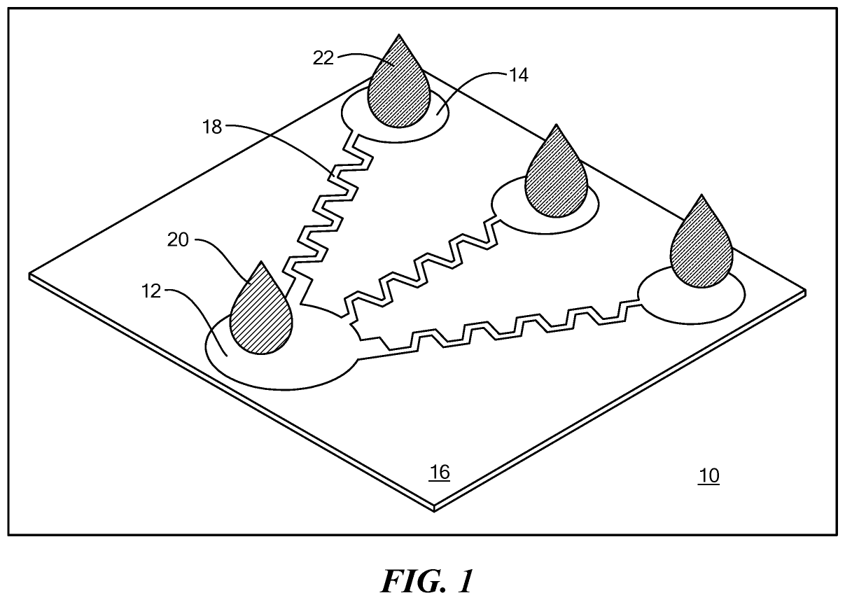

FIG. 1 shows a microfluidic paper-based analytical device 10 that comprises a sampling zone 12 and a detection zone 14 disposed on a substrate 16. A transport channel 18 extends between the sampling zone 12 and the detection zone 14. The sampling zone 12, detection zone 14, and transport channel 18 comprise a material that promotes flow by capillary action. A suitable material is paper.

In a preferred embodiment, the sampling zone 12 and the detection zone 14 are circular regions having diameters having average values of eight millimeters and six millimeters, respectively. In some embodiments, the transport channel 18 is a straight channel eleven millimeters long and two millimeters wide.

The sampling zone 12 is pre-loaded with a nanozyme 20 and the detection zone 14 is pre-loaded with a reagent 22. In a preferred embodiment, the nanozyme 20 comprises gold nanoparticles and the reagent comprises silver nanoparticles. Suitable nanoparticles are spherical nanoparticles with diameters having average values of ten and eight nanometers for gold and silver, respectively.

The first nanozyme 20 acts as a catalyst to promo of glucose. Among the products of this oxidation is hydrogen peroxide. In those cases in which the nanozyme comprises gold nanoparticles, the glucose is oxidized into gluconic acid and hydrogen peroxide. This hydrogen peroxide flows towards the detection zone 14.

When the hydrogen peroxide reaches the detection zone 14, it interacts with the reagent 22. This interaction results in a color change, and in particular, a change from light yellow to clear. The extent of this color change depends on the hydrogen peroxide concentration.

In the case in which the reagent comprises silver nanoparticles, the hydrogen peroxide etches the silver nanoparticles to form a silver ion, a hydroxyl ion, and a hydroxyl radical. This etching results in a perceptible color change.

Since the hydrogen peroxide was originally derived from glucose, the hydrogen peroxide concentration depends on the original glucose concentration. As a result, the extent of the color change at the detection zone 14 ultimately depends on the concentration of glucose. This makes it possible to assay the glucose concentration based on the extent of the color change at the detection zone 14.

An unexpected advantage is that the reaction time on a solid substrate, such as paper, turned out to be significantly faster than a similar reaction in a purely liquid phase. This faster reaction time means a shorter wait for the results of a glucose assay.

In principle, only one detection zone 14 is needed. However, to promote greater accuracy, it is useful to have plural detection zones 14 to obtain an average color. In addition, it some embodiments, the different detection zones 14 are pre-loaded with slightly different reagents 22. Among these are embodiments in which the reagents 22 have silver nanoparticles that differ slightly in size from one detection zone 14 to the next to accommodate detectors with different color sensitivity profiles.

A suitable method for preparing the silver nanoparticles is to mix fifty milliliters of silver nitrate solution at a concentration of 0.3 millimoles per liter with four milliliters of sodium citrate solution at a concentration of 12.6 millimoles per liter. This is followed by a rapid injection of one milliliter of sodium borohydride solution with vigorous stirring. This results in the previously colorless solution acquiring a yellow tint.

A suitable method of preparing gold nanoparticles is to heat fifty milliliters of chloroauric acid at a concentration of forty millimoles per liter. The solution is heated and stirred for about two hours. This is followed by dropping two milliliters of 1.0% (w/v) sodium citrate into the solution while heating and stirring. In about half an hour, the solution develops a deep red color similar to red wine. This is followed by injection of 0.1 milliliters of cysteine at a concentration of fifteen micromoles per liter into the solution to decorate the gold nanoparticles.

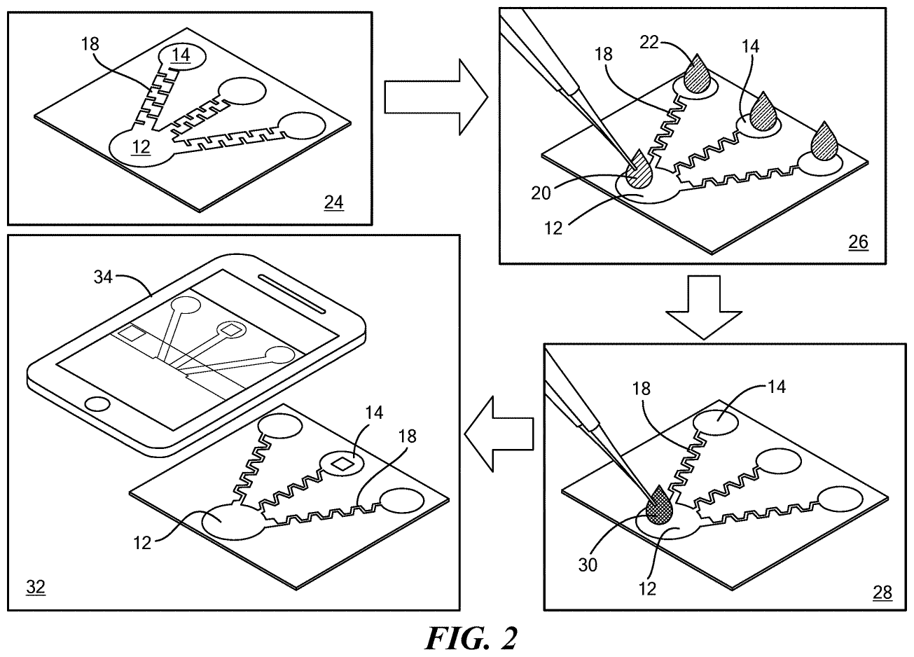

FIG. 2 shows the process of manufacturing and then using the device 10.

In a first step 24, a wax printer prints, on a piece of filter paper, a design corresponding to the layout of the sample region 12, detection region 14, and transport channel 18. The resulting device 10 is then heated to about 120° C. for two minutes and cooled to room temperature. The back side of the resulting device 10 is then sealed with adhesive tape to prevent the solution from leaking through the device 10.

In a second step 26, the device 10 is loaded by adding five microliters of a solution containing the nanozyme 20 to the sample zone 12 and three microliters of a solution containing the reagent to the detection zone 14. The device 10, now having been loaded, is left to dry at room temperature.

In a third step 28, a sample 30 of bodily fluid, such as saliva, is dropped onto the sample zone 12. The nanozyme 22 catalyzes an oxidation reaction that results in hydrogen peroxide.

This hydrogen peroxide flows by capillary action to the detection zone 14 via the transport channel 18.

A fourth step 32 includes estimating glucose concentration by inspecting of the color change that resulted from interaction of the reagent 22 with the hydrogen peroxide. Although this can be carried out visually, to promote more accurate detection, it is useful to provide an optical detector 34 that separates input light into red, green, and blue channels. A suitable detector is a smartphone that has a camera and color-analysis software executing thereon.

Having described the invention and a preferred embodiment thereof, what is claimed as new and secured by letters patent is:

1. An apparatus comprising an assay device made of paper, wherein said assay device comprises a sampling zone, a detection zone, and a transport channel extending therebetween to support capillary flow between said sampling zone and said detection zone, a nanozyme on said sampling zone, and a reagent on said detection zone, wherein said sampling zone, said detection zone, and said transport channel comprise said paper, wherein said nanozyme comprises nanoparticles that comprise a first metal, wherein said reagent comprises nanoparticles that comprise a second metal, and wherein said nanoparticles of said second metal change to a color that corresponds to concentration of an analyte deposited onto said sample zone.

2. The apparatus of claim 1, wherein said analyte is glucose, wherein said nanozyme comprises gold nanoparticles, wherein said reagent comprises silver nanoparticles, wherein said color change is a change from yellow to a color outside the visible range of light, and wherein said silver nanoparticles react with hydrogen peroxide produced as a result of oxidation of glucose by said gold nanoparticles.

3. The apparatus of claim 1, further comprising a sensor that detects said color and provides an estimate of a concentration of said analyte based on said color.

4. The apparatus of claim 1, wherein said nanozyme comprises metallic nanoparticles that catalyze a reaction with glucose and wherein said reagent comprises metallic nanoparticles that react with a product of the reaction.

5. The apparatus of claim 1, wherein said analyte is glucose, wherein said nanozyme catalyzes oxidation of said glucose, and wherein said reagent reacts with a product of said oxidation.

6. The apparatus of claim 1, further comprising a smartphone having a camera, said smart phone having been configured to carry out colorimetric analysis of an image captured by said camera.

7. The apparatus of claim 1, wherein said detection zone is one of a plurality of detection zones, wherein said transport channel is one of a plurality of channels, each of which connects said sampling zone to a corresponding one of said detection zones.

8. The apparatus of claim 1, wherein said transport path is a meandering transport path.

9. The apparatus of claim 1, wherein said nanozyme comprises gold nanoparticles.

10. The apparatus of claim 1, wherein said reagent comprises silver nanoparticles.

11. The apparatus of claim 1, wherein said analyte is glucose.

12. The apparatus of claim 1, wherein said analyte is glucose, said nanozyme comprises gold particles, and said reagent comprises silver nanoparticles.

13. The apparatus of claim 1, wherein said nanozyme comprises platinum nanoparticles.

14. The apparatus of claim 1, wherein said reagent comprises nickel nanoparticles.

15. The apparatus of claim 1, wherein said reagent comprises nickel-ferrite nanoparticles.

16. The apparatus of claim 1, wherein said nanozyme comprises gold nanoparticles that promote oxidation of glucose into gluconic acid and hydrogen peroxide and wherein said hydrogen peroxide and said reagent interact to cause a color change that depends on glucose concentration.

17. The apparatus of claim 1, wherein said reagent is configured to react with a product of a reaction that was catalyzed by said nanozyme, wherein said analyte was a reagent in said reaction.

18. A method comprising using an assay device made of paper, wherein said assay device comprises a sampling zone, a detection zone, and a transport channel extending therebetween, a nanozyme on said sampling zone, and a reagent on said detection zone, wherein said sampling zone, said detection zone, and said transport channel comprise paper, wherein said nanozyme comprises nanoparticles of a first metal, and wherein said reagent comprises nanoparticles of a second metal, said nanoparticles having been selected to change to a color that corresponds to concentration of an analyte deposited onto said sample zone, wherein using said assay device comprises placing a sample of a bodily fluid on said sampling zone, thereby initiating a reaction that is catalyzed by said nanozyme and causing a reaction product thereof to engage in capillary flow along said transport channel towards said detection zone, wherein said reaction product interacts with said reagent at said detection zone to cause a color change, and estimating a concentration of said analyte in said sample.

19. A method comprising manufacturing an assay device made of paper, wherein said assay device comprises a sampling zone, a detection zone, and a transport channel extending therebetween to support capillary flow between said sampling zone and said detection zone, a nanozyme on said sampling zone, and a reagent on said detection zone, wherein said sampling zone, said detection zone, and said transport channel comprise said paper, wherein said nanozyme comprises nanoparticles of a first metal, and wherein said reagent comprises nanoparticles of a second metal, said nanoparticles of said second metal having been selected to change to a color that corresponds to concentration of an analyte deposited onto said sample zone, wherein manufacturing said assay device comprises using a wax printer, printing, on a piece of said paper, said sample region, said detection region, and said transport channel, said method further comprising pre-loading said sample region with said nanozyme, and pre-loading said detection region with said reagent.
